(19) United States
(12) Patent Application Publication
(10) Pub. No.: US 2008/0058596 A1
(43) Pub. Date: Mar. 6, 2008

(54) EVERTING TUBE COMPRISING TWO-COMPONENT LUBRICANT

(75) Inventors: Konstantin Bob, Weinheim (DE); Fritz Pauker, Kissing (DE); Thomas Viebach, Pischershofen (DE)

Correspondence Address: STANDLEY LAW GROUP LLP, 495 METRO PLACE SOUTH, SUITE 210, DUBLIN, OH, 43017, US

(73) Assignee: invendo medical GmbH, Weinheim (DE)

(21) Appl. No.: 11/846,636

(22) Filed: Aug. 29, 2007

(30) Foreign Application Priority Data
Sep. 1, 2006 (DE) 102006000435.3

(51) Int. Cl. A61B 1/00

(52) U.S. Cl. 600/114

(57) ABSTRACT

A medico-technical device, such as an endoscope (1), has an everting tube (3) that encloses an endoscope shaft (2) for driving the latter. To minimize friction incurred by relative movement between the endoscope shaft (2) and the everting tube (3) and/or friction between two opposed surfaces of the everting tube (3), a lubricant is applied to the areas. To accomplish this, at least one of the everting tube (3) and the endoscope shaft (2) are coated with a first component provided in solid matter. By adding a second component, which reacts with the first component, to the first component, the first component liquefies and/or swells so as to develop its lubricating properties.

### CROSS-REFERENCE TO RELATED APPLICATIONS

This application claims a benefit of priority to German application 102006000435.3, filed 1 Sep. 2006, which is incorporated by reference as if fully recited herein.

### TECHNICAL FIELD

The present invention relates to a medico-technical device which can be made to intervene with a human or animal body, and more exactly speaking to an endoscope.

### BACKGROUND OF THE ART

Endoscopic devices as well as devices for introducing a medical endoscope into a body canal are described, for instance, in U.S. Pat. No. 5,259,364. The devices described there permit that an endoscope is no longer pushed into the body to be examined but moves into the body by itself. For this purpose, the endoscope is equipped with a drive (inherent drive) which permits easier and quicker insertion.

As such an inherent drive, for instance, also a so-called everting tube can be used into which the endoscope shaft is introduced. Upon propulsion of the endoscope different relative movements occur. On the one hand, a relative movement occurs between the endoscope shaft and the everting tube which are in sliding contact with each other. On the other hand, also a relative movement between an inside portion and an outside portion of the unwinding everting tube occurs.

In order to reduce the sliding friction occurring in each case as well as an adhesion between the respective elements, for example the use of a lubricant supplied from outside was suggested, e.g. in U.S. Pat. No. 6,077,219. In both cases a lubricant is supplied between the endoscope shaft and the everting tube, on the one hand, and between the two superposed everting tube portions, on the other hand. The lubricants used in most cases are oils, such as e.g. edible oils. In the case of oils there is the problem, however, that they can migrate e.g. along the endoscope shaft and can cover the lens of the endoscope with a lubricating film which is difficult to remove.

Accordingly, it is the object of the present invention to provide an improved medico-technical device, especially an improved endoscopic device. Specific improvements which are preferably to be achieved are especially a simplified structure of the medico-technical device, increased efficiency, enhanced acceptance with the individual in whom the device is used as well as a reduction of the frictional forces occurring in the medico-technical device.

### SUMMARY OF THE INVENTION

In accordance with the invention, the object is achieved by a medico-technical device according to the claims appended hereto.

Further advantageous configurations of the invention are the subject matter of the subclaims.

Accordingly, the core of the invention consists in the fact that a medico-technical device, such as, for instance, an endoscope, includes at least one element provided with a first component which develops lubricating properties only when a second component is added. That is to say an element exposed to friction during operation of the medico-technical device such as, e.g., an everting tube or an endoscope shaft/endoscope shaft cover of an endoscope, is in advance coated with a first component in solid matter, such as, e.g., cellulose, which is liquefied by a second component, such as water, when putting the medico-technical device or the endoscope into operation so as to develop lubricating properties in this way. Thus a friction between the element exposed to friction during operation and elements which are in frictional contact with said element is reduced.

The medico-technical device preferably is an endoscope.

Advantageously the element is a portion or a part of the medico-technical device which is exposed to strong friction during operation of the medico-technical device. The element preferably is an everting tube and/or an endoscope shaft/endoscope shaft cover of an endoscope. In this way an adhesion and sliding friction between an everting tube and an endoscope shaft/an endoscope shaft cover of the endoscope and/or between the surfaces of the everting tube moving relative to each other can be reduced. The element can also be any other part or a portion of a part of the medico-technical device which is exposed to friction during operation of the endoscopic device.

In general, the first component is provided at least at a part of an outer surface and/or an inner surface of the element. As an alternative, also the entire surface of the element (both outer surface and inner surface) can be provided with the first component, however. Preferably both the entire inner and outer surfaces of the everting tube are provided with the first component.

In a preferred embodiment the first component has a high bonding energy with respect to the element before addition of the second component. Advantageously, the bonding energy is so high that the first component is efficiently prevented from detaching from the element, especially in places of strong deformation of the element, such as e.g. the everting points of an everting tube. In this context, it is also desirable that before addition of the second component the first component moreover has a high flexural elasticity.

It is also desired that before addition of the second component the first component is provided in solid matter, so that a possible drying, e.g. when the medico-technical device or the element is stored for a long time, does not have to be taken into consideration. This is no necessary prerequisite for the subject matter of the invention, however.

Advantageously, the first component is accommodated in cavities formed in the everting tube. Said cavities can be perforations, grooves etc.

The cavities can be produced by laser engraving. However, any other method is applicable by which it is possible to form cavities in the everting tube, such as e.g. embossing, foaming, cutting, rolling etc.

The cavities are preferably formed by a plasma treatment.

Advantageously, the element of the medico-technical device is made of rubber, preferably of silicone rubber.

The first and/or second component is/are advantageously made of a physically tolerated/biocompatible or biologically degradable material.

Macromolecules capable of swelling are advantageously used as first component. Preferably the first component is a polysaccharide. In this case, the first component advantageously consists of starch, glycogen, cellulose, recombinant cellulose or agar-agar. It is preferred that the first component consists of an alginate with soybean protein additive and/or whey protein additive.

As an alternative, the first component can be a peptide. In this case, the first component advantageously consists of collagen, gelatin or recombinant gelatin.

Moreover, the first component may also be a synthetic material or a silicate.

The second component preferably is oil, water or an oil-water emulsion.

Alternatively, also body fluid, such as fluid contained in the intestines, could be used as second component.

Furthermore, it is possible that the second component consists of alcohols, such as e.g. ethanol, glycol, propanol or glycerin.

It is preferred that the medico-technical device is an endoscope comprising an endoscope shaft which can include an endoscope shaft cover and an everting tube enclosing the endoscope shaft at least partly.

Advantageously the second component is applied to places of the medico-technical device which are exposed to strong friction during operation of the medico-technical device. In the endoscope the second component is preferably applied to a front eversion and/or a rear eversion of the everting tube. The second component may also be applied, however, in addition or as an alternative between the endoscope shaft and the everting tube. But it is also possible to apply the second component additionally or alternatively between everted surfaces of the everting tube and/or on a surface of the everting tube facing away from the endoscope shaft.

The second component is preferably supplied to the first component through a conduit/channel formed in the endoscope shaft. Said conduit may be, for instance, a working conduit, a coolant conduit or another conduit formed in the endoscope. The second component needs not necessarily be supplied through a conduit formed in the endoscope, however, but can be supplied, for instance, through an externally formed conduit.

Advantageously, the endoscope moreover includes an external supply chamber in which the second component is stored and from which the second component is supplied.

### BRIEF DESCRIPTION OF THE DRAWINGS

Hereinafter the invention will be explained in detail by way of a preferred embodiment with reference to the accompanying drawing, in which:

FIG. 1 schematically shows the structure of an endoscope comprising an everting tube according to a preferred embodiment of the present invention.

### DETAILED DESCRIPTION OF A PREFERRED EMBODIMENT

Although in this embodiment only the everting tube is provided with the first component which develops its lubricating properties only when activated by a second component, it should be clear that other and/or additional elements of the endoscope can be provided with the first component.

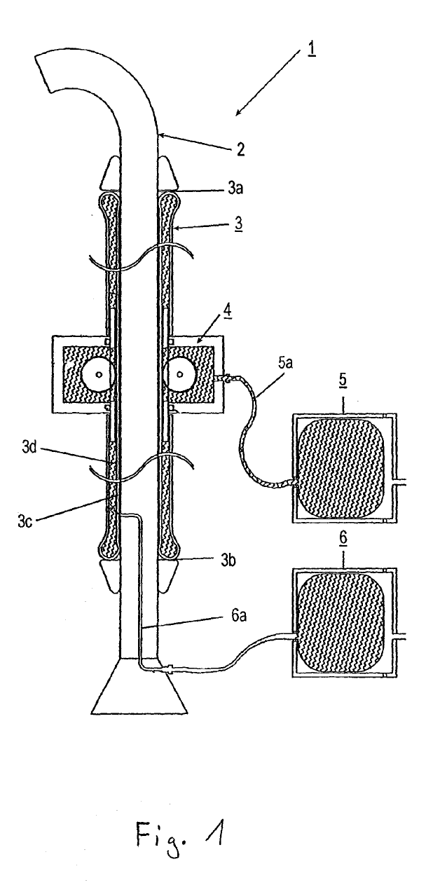

As it is illustrated in FIG. 1, the endoscope 1 substantially comprises an endoscope shaft 2, an everting tube 3 and a drive means 4. In addition, in this embodiment two external reservoir chambers 5 and 6 are provided which are in fluid communication through lines 5a and 6a with an inside 3d and outside 3a, 3b, 3c, respectively, of the everting tube 3.

According to the invention, the everting tube 3 is provided with a first component having lubricating properties only when activated by or interacting with a second component, i.e. the lubricant itself is not formed before the first component is activated by the second component.

In this way the everting tube 3 can be provided with the first component, the actual lubricant, already during manufacture, without the lubricant losing its lubricating properties, because it develops the same only when activated by the second component. In contrast to everting tubes which are coated with oil before their actual use, the advantage is that during storage or before the actual use of the everting tube, which is part of a medical device, no lubricant can escape or migrate from the everting tube to other parts of the medico-technical device.

Preferably, before activation by the second component the first component has a solid form the properties of which are not impaired even during long-time storage.

The first component moreover is preferably applied both to the entire inner surface 3d and to the entire outer surface 3c of the everting tube. Preferably, the first component is applied in a homogenous manner.

Applying the first component to the everting tube 3 can be realized by any suitable conventional method, such as e.g. dip coating, roller coating, blade coating, spin coating, the so-called doctor blade method etc. Advantageously, by the method employed a uniform and thus particularly smooth application of the first component is achieved.

The everting tube 3 is subjected to strong deformation especially at its everting points. Therefore, the first component advantageously has a high bonding energy with respect to the everting tube 3 as well as a high flexural elasticity in order to ensure that the first component does not detach from the everting tube 3. Also with regard to activation it is desirable that after activation by the second component the first component has a sufficiently high bonding energy with regard to the everting tube so that the lubricant formed in this way remains in the provided places and does not migrate or migrates in as small quantities as possible into the examined human or animal body. In order to increase the bonding energy of the first component with respect to the everting tube 3, it is possible, for instance, to roughen the surface of the everting tube or provide it with grooves so as to permit a better adhesion of the first component. In this context, also a chemical preparatory treatment is imaginable.

In order to ensure an especially good segregation of the lubricant formed as well as storage of the first component directly on the surface of the everting tube 3 it is preferred that at least one surface of the everting tube 3 shows a profile the cavities of which contain the first component. The cavities, e.g. in the form of grooves, can be advantageously produced by embossing, foaming and cutting open, cutting or rolling or by engraving, especially laser engraving.

In a preferred embodiment of the invention the first component therefore is provided in grooves disposed directly on a surface of the everting tube 3. In some embodiments the profile is formed by regularly arranged grooves, in other embodiments by irregularly arranged grooves.

As an alternative, the everting tube can have a layered structure. The layered structure comprises at least one substrate layer so as to form a stable basis for layers arranged thereon, a reservoir layer arranged on at least one, preferably both sides of the substrate for the first component so as to store sufficient lubricant for the actual use and at least one or, if on both sides of the substrate a reservoir layer is arranged, two external perforated layer(s) so as to release the lubricant, i.e. the first component after activation by the second component, in a controlled manner. In the first case the basic structure of substrate/reservoir layer/perforated layer is resulting, in the second case the basic structure of perforated layer/reservoir layer/substrate/reservoir layer/perforated layer is resulting, wherein intermediate layers can further be provided in each case.

The substrate layer is formed by the everting tube 3 itself, the latter being advantageously made of silicone rubber. The reservoir layer to advantage is a layer consisting of the first component which is preferably provided in solid matter. By activating said layer by the second component the first component is liquefied and swells, respectively, i.e. it develops its lubricating properties, and the lubricant formed in this way is subsequently released to at least one adjacent layer.

It is also possible that the reservoir layer is formed to have a sponge-like structure or by a material having a capillary effect, wherein the first component is sucked by the sponge-like structure or by the material and, in turn, is released to at least one adjacent layer by activation by the second component.

In case that two reservoir layers are provided, they can be formed identically or differently. A different configuration can be especially advantageous, if different pressure impacts act upon the everting tube 3. In the everting tube 3 different physical influences, such as e.g. pressure in the internal and the external reservoir layers occur particularly in the everting area so that a different configuration of this layer which is adapted to the respective external conditions may be useful. The same consideration is also applicable to the perforated layer described hereinafter.

Preferably the perforated layer is provided to be directly adjacent to the reservoir layer and forms the outer surface of the element. However, between the reservoir layer and the perforated layer further layers may be arranged as long as they are substantially permeable for the first component and the lubricant formed by activation by the second component, respectively. The perforation of the perforated layer may have any design. It is preferred that this layer has through-holes or conduits the central axes of which enclose an almost right angle, i.e. of e.g. 80 to 100°, especially approx. 90°, with the surface of the element the surface of which is formed by the perforated layer. In this way, the lubricant formed migrates from the reservoir layer to the surface to be lubricated in a particularly efficient and controlled manner.

The element built up in layers permits a simple manufacture by preparing e.g. the individual layers as required in advance and then laminating them together or by applying the lubricant layer to the substrate (everting tube 3) or the uppermost of the layers possibly arranged above the substrate and said composite is then provided with a perforated layer. It is an advantage of this configuration that the lubricant reservoir or, rather, the reservoir for the first component can be designed with any parameters depending on the application.

The aforementioned profile is preferably adapted such that released lubricant is supplied to the areas exposed to especially high friction. To this effect, the profile can have longitudinally, transversely or spirally extending cavities which are preferably constituted by grooves or slots or by individual locally confined indentations.

A profile suited for the purpose of accommodating the first component and releasing lubricant in a controlled manner can be produced by engraving, especially by laser engraving. An everting tube 3 having a profile to be produced by laser engraving is therefore likewise included in the present invention.

Laser engraving permits a controlled material abrasion which takes place, for instance, by means of a pulsed laser beam. The beam is preferably very strongly focused in order to improve the machining accuracy, wherein e.g. focal points having a diameter of 0.01 to 0.1 mm are generated. In the focal point very high temperatures of more than 2000° C., for instance, can be reached, which permits a rapid machining of almost any materials.

During an engraving step warps may occur on the treated surface laterally from the cavities produced. Such warps reduce the sliding properties of the everting tube 3. Therefore it is further preferred that the profile can be obtained by laser engraving and subsequent smoothing.

Ceramic coating, silicatizing, UV irradiation, acrylate coating and polysilazane coating have turned out to be especially suited methods for smoothing the engraved surface so that the warps are leveled and at the same time a depth of the engraved cavities suited for receiving a sufficient quantity of the first component is retained.

According to the invention, it is moreover possible to employ one of the afore-mentioned methods for surface treatment without preceding laser engraving. In this case a layer of the first component is generated on the surface.

Another preferred alternative for forming cavities in the everting tube for receiving the first component is the use of a conventionally known plasma treatment, such as e.g. used by the Fraunhofer institute for boundary surfaces and process engineering. Such a plasma method serves in its known application for roughening or activating the surface of a substrate in order to increase the adhesive characteristics of the substrate. The applicant found out, however, that the cavities formed by the plasma method are sufficient to be capable of introducing the first component (preferably an alginate) and that the lubricating effect to be reached thereby is sufficient for a satisfactory lubrication of the everting tube and the endoscope shaft/endoscope shaft cover, respectively. Therefore the conventionally known plasma method is used, apart from its original purpose, for producing cavities in which the first component is accommodated. In this context, the use of an alginate as a first component is preferred.

As the everting tube 3 is preferably used for an endoscope, it is advantageous that the first component and the second component are physically tolerated/biocompatible and/or are such that they are innocuous to the body to be examined in the quantities used. The first and second components may also consist of biologically degradable materials.

In accordance with the invention, the first component develops its lubricating component or the first component swells only after being activated by a second component.

As first component the use of polysaccharides such as starch, glycogen, cellulose, recombinant cellulose (obtained, e.g., from microorganisms or transgenic plants), agar-agar or an alginate with soybean protein additive and/or starch protein additive is especially preferred. These elements can be formed very easily in solid form as a layer and moreover are particularly physically tolerated. As an alternative, also peptides such as, e.g., collagen, gelatin or recombinant gelatin (obtained e.g. from microorganisms or transgenic plants) are very well suited for use as a first component, because they, too, can be formed especially easily as a layer and have an especially good physical tolerance. Furthermore, also other macromolecules capable of swelling or else plastic materials can be taken into consideration for such use.

When using the afore-described elements as first component, the use of water as second component is of special advantage, because the latter is generally available and physically tolerated. When activated by water, the above-mentioned elements liquefy and/or swell and thus develop their lubricating properties. It is also possible, however, e.g. when using the everting tube in an endoscopic device, to use body fluid provided in the area to be examined as the second component. For use as second component also alcohols, such as e.g. ethanol, glycol, propanol or glycerin are very well suited.

When using the afore-mentioned first and second components for forming a lubricant, it is to advantage, in contrast to oils which, if they migrate, for instance, to a lens of an endoscope, form a fatty, viscous lubricating film on the same which is difficult to remove, that the lubricant can be very easily removed from the lens, e.g. by a weak water jet.

With reference to FIG. 1, the second component is stored in two reservoirs (5, 6) from which, for activating the first component, the second component is conveyed via lines 5a and 6a to the everting tube on which the first component is provided. The line 5a is connected to the housing of the drive 4 in order to apply the second component to the inner surface 3d of the everting tube 3. The line 6a leads through the endoscope shaft 2 and opens at an outside of the endoscope shaft 2 opposed to an outside 3c of the everting tube 3. Thus the second component is applied to both the inside 3d and the outside 3a, 3b, 3c of the everting tube.

The invention is not restricted to the afore-described arrangement but is subject to plural modifications. It is mainly important that the second component is safely supplied to an area of the element/everting tube to which the first component is applied. Accordingly, only a common reservoir may be formed, for instance, from which two lines branch off or, for example when using body fluid, reservoirs and/or lines can be completely dispensed with. It is also possible, however, that the second component is only supplied via a line which either extends through the endoscope shaft or is formed separately from the endoscope shaft. If use is made of a line extending through the endoscope shaft, this line needs not be newly provided, but existing lines or conduits, such as a working conduit, cooling conduit or a conduit for spraying-nozzle supply, can be used partly or completely.

In the embodiment shown in FIG. 1 the second component is applied to the inside 3d of the everting tube 3 and to a place at the outside 3c of the everting tube between the everting tube 3 and the endoscope shaft 2. However, the invention is not restricted to such an arrangement. Preferably the second component is applied to areas on the everting tube which are exposed to especially high friction. So it is imaginable that the second component is applied, e.g., to one or both everting points 3a, 3b of the everting tube.

A medico-technical device, such as an endoscope, has an everting tube which encloses an endoscope shaft for driving the same. In order to minimize friction due to a relative movement between the endoscope shaft and the everting tube and/or friction between two opposed surfaces of the everting tube, a lubricant is applied to the said areas. For this purpose, the everting tube and/or the endoscope shaft are coated with a first component provided in solid matter. By adding a second component, which reacts with the first component, to the first component, the first component liquefies and/or swells so as to develop its lubricating properties.

What is claimed is:

1. A medico-technical device comprising:
at least one element provided with a first component which develops lubricating properties only when a second component is added.

2. The medico-technical device according to claim 1, wherein:
the element is at least one of: an everting tube, an endoscope shaft and an endoscope shaft cover.

3. The medico-technical device according to claim 1, wherein:
the first component is provided in solid matter before the second component is added.

4. The medico-technical device according to claim 1, wherein:
the first component has a high bonding energy with respect to the element.

5. The medico-technical device according to claim 1, wherein:
the first component exhibits a high flexural elasticity.

6. The medico-technical device according to claim 1, wherein:
the first component is accommodated in cavities formed in the surface of the element.

7. The medico-technical device according to claim 1, wherein:
the first component and the second component are made of a physically tolerated and/or biologically degradable material.

8. The medico-technical device according to claim 1, wherein:
the first component is a polysaccharide.

9. The medico-technical device according to claim 1, wherein:
the first component comprises starch, glycogen, cellulose, recombinant cellulose, agar-agar or an alginate with soybean protein additive and/or whey protein additive.

10. The medico-technical device according to claim 13, wherein:
the first component comprises collagen, gelatin or recombinant gelatin.

11. The medico-technical device according to claim 1, wherein:
the second component is at least one of: oil, water, body fluid and an emulsion of oil and water.

12. The medico-technical device according to claim 2, wherein:
the second component is applied to at least one of a front eversion and a rear eversion of the everting tube.

13. A method of providing a medico-technical device that develops lubricating properties, the method comprising the steps of:
providing an element on the medico-technical device;
producing cavities on a surface of the element by means of a plasma treatment; and
introducing a first component into the cavities produced;
wherein the first component is activated as a lubricant only upon application of a second component.

14. The method according to claim 13, wherein:
the element is at least one of: an everting tube, an endoscope shaft, and an endoscope shaft cover.

15. The method according to claim 13, wherein:
the first component comprises starch, glycogen, cellulose, recombinant cellulose, agar-agar or an alginate with soybean protein additive and/or whey protein additive.

16. The method according to claim 13, wherein:
the first component comprises collagen, gelatin or recombinant gelatin.

17. The method according to claim 13, wherein:
the second component is at least one of: oil, water, body fluid and an emulsion of oil and water.
